(19) United States
(12) Patent Application Publication
(10) Pub. No.: US 2022/0401051 A1
(43) Pub. Date: Dec. 22, 2022

(54) RADIATION IMAGING SYSTEM, IMAGE PROCESSING APPARATUS, IMAGE PROCESSING METHOD, AND NON-TRANSITORY COMPUTER READABLE STORAGE MEDIUM

(71) Applicant: CANON KABUSHIKI KAISHA, Tokyo (JP)

(72) Inventors: Sota Torii, Tokyo (JP); Takeshi Noda, Kanagawa (JP)

(21) Appl. No.: 17/743,525

(22) Filed: May 13, 2022

(30) Foreign Application Priority Data
Jun. 16, 2021 (JP) 2021-100431

(51) Int. Cl. A61B 6/00; H04N 5/32

(52) U.S. Cl. CPC A61B 6/5258; H04N 5/32; A61B 6/5294

(57) ABSTRACT

A radiation imaging system comprises: an image obtaining unit including a radiation detecting unit in which pixels configured to output signals according to a dose of irradiated radiation are arranged in a two-dimensional area, and configured to obtain a radiation image based on the signals; a correction unit configured to correct the radiation image using an input/output characteristic of a pixel, which represents a relationship between the dose of radiation on the pixel and the signal output from the pixel and is obtained using gain data based on a plurality of gain images obtained under different doses; and an updating unit configured to update the gain data using an updating coefficient obtained based on the gain data and a gain image newly obtained by the image obtaining unit.

### BACKGROUND OF THE INVENTION

### Field of the Invention

The present invention relates to a radiation imaging system, an image processing apparatus, an image processing method, and a non-transitory computer readable storage medium and, more particularly, to a radiation imaging system, an image processing apparatus, an image processing method, and a non-transitory computer readable storage medium, which are preferably used for still image capturing such as general imaging or moving image capturing such as fluoroscopic imaging in a medical diagnosis.

### Description of the Related Art

In recent years, a flat panel detector (to be referred to as an “FPD” hereinafter) formed by two-dimensionally arraying solid-state imaging elements made of amorphous silicon or single-crystal silicon and configured to capture a radiation image has widely been put into practical use.

Since the FPD is formed by a plurality of solid-state imaging elements, the input/output characteristic changes between the solid-state imaging elements. To align the different input/output characteristics between the solid-state imaging elements, a gain image is captured before imaging, and gain correction is performed for a captured image, thereby correcting a radiation image using the input/output characteristics of the solid-state imaging elements.

Here, if there is only gain data of one point representing a dose and the input/output characteristic of a pixel value, it is necessary to linearly approximate the input/output characteristic under a different dose using a linear function and correct. However, if the input/output characteristic is nonlinear, the deviation part from the linear approximation may be generated as an artifact in an image. Japanese Patent No. 6674222 discloses a technique for executing gain correction by obtaining a plurality of gain images under different doses and approximating the input/output characteristic of a pixel by a nonlinear function to reduce generation of such an artifact.

However, an obtained gain image cannot be used permanently. Since the irradiation distribution of an X-ray tube or light emission of phosphor changes over time, it is necessary to reobtain a gain image in, for example, apparatus maintenance conducted every predetermined period. For this reason, if a plurality of gain images captured under different dose conditions are obtained. every time maintenance is performed, as in Japanese Patent No. 6674222, the maintenance man-hours may increase. The present invention has been made in consideration of the above-described problem, and provides a technique capable of reducing a man-hours needed to obtain a plurality of gain images.

### SUMMARY OF THE INVENTION

According to one aspect of the present invention, there is provided a radiation imaging system comprising: an image obtaining unit including a radiation detecting unit in which pixels configured to output signals according to a dose of irradiated radiation are arranged in a two-dimensional area, and configured to obtain a radiation image based on the signals; a correction unit configured to correct the radiation image using an input/output characteristic of a pixel, which represents a relationship between the dose of radiation on the pixel and the signal output from the pixel and is obtained using gain data based on a plurality of gain images obtained under different doses; and an updating unit configured to update the gain data using an updating coefficient obtained based on the gain data and a gain image newly obtained by the image obtaining unit.

Further features of the present invention will become apparent from the following description of exemplary embodiments (with reference to the attached drawings).

### BRIEF DESCRIPTION OF THE DRAWINGS

FIG. 1 is a block diagram showing the configuration of a radiation imaging system according to an embodiment of the present invention;

FIG. 2 is a flowchart for explaining the procedure of processing of estimating gain coefficients to be used in multi-point gain correction according to the first embodiment;

FIG. 3 is a graph exemplarily showing changes of pixel values caused by multi-point gain correction using a polynomial function whose gain coefficients are updated based on an updating coefficient;

FIG. 4 is a flowchart for explaining the procedure of processing of estimating gain coefficients to be used in multi-point gain correction according to the second embodiment; and

FIG. 5 is a graph schematically showing the input/output characteristic of a pixel in each dose region according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted. In the following embodiments and claims, radiation includes not only X-rays but also α-rays, β-rays, γ-rays, and various kinds of particle beams.

### First Embodiment

### Configuration of Radiation Imaging System 100

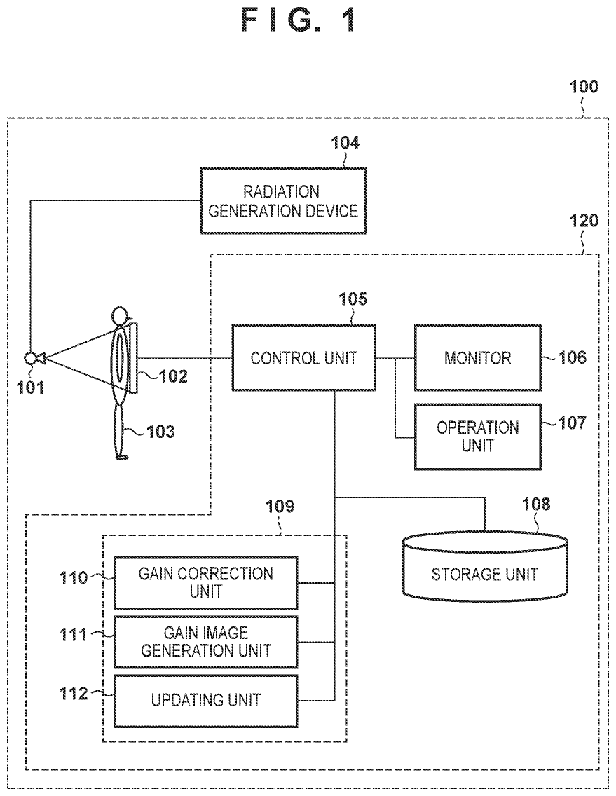

FIG. 1 is a block diagram showing an example of the configuration of a radiation imaging system 100 according to the first embodiment of the present invention. The radiation imaging system 100 includes a radiation generation device 104, a radiation tube 101, an FPD 102 (radiation detector), and an image processing apparatus 120. Note that the configuration of the radiation imaging system 100 is also simply called a radiation imaging apparatus. The image processing apparatus 120 processes information (image information) based on a captured radiation image.

When an exposure switch is pressed, the radiation generation device 104 applies high-voltage pulse to the radiation tube 101 to generate X-rays, and the radiation tube 101 irradiates an object 103 with the radiation. The type of radiation is not particularly limited. In general, X-rays are used, as used here.

When the radiation tube 101 irradiates the object 103 with the radiation, the FPD 102 functions as an image obtaining unit, accumulates charges based on an image signal, and obtains a radiation image. The FPD 102 includes a radiation detecting unit (not shown) including a pixel array configured to generate (output) a signal according to the dose of irradiated radiation. The radiation detecting unit detects radiation transmitted through the object 103 as an image signal. In the radiation detecting unit, pixels configured to output signals according to incident light are arranged in an array (two-dimensional area). The photoelectric conversion element of each pixel converts radiation, which is converted by phosphor into visible light, into an electrical signal, and outputs it as an image signal. As described above, the radiation detecting unit is configured to detect radiation transmitted through the object 103 and obtain an image signal (radiation image).

The drive unit of the FPD 102 outputs, to a control unit 105, an image signal (radiation image) read out in accordance with an instruction from the control unit 105 of the image processing apparatus 120.

The image processing apparatus 120 processes information (image information) based on a captured radiation image. The image processing apparatus 120 includes the control unit 105, a monitor 106, an operation unit 107, a storage unit 108, an image processing unit 109, and a display control unit 116.

The control unit 105 includes one or a plurality of processors (not shown), and executes programs stored in the storage unit 108, thereby implementing various kinds of control of the image processing apparatus 120. The storage unit 108 stores results of image processing and various kinds of programs. The storage unit 108 is formed by, for example, a ROM (Read Only Memory), a RAM (Random Access Memory), or the like. The storage unit 108 can store an image output from the control unit 105, an image processed by the image processing unit 109, and a calculation result in the image processing unit 109. Also, the storage unit 108 holds gain data obtained in advance under different doses. Here, the gain data includes gain coefficients (to be referred to as “gain data (gain coefficients)” hereinafter) obtained based on a plurality of gain images obtained in advance under different doses, and pixel values (to be referred to as “gain data (pixel values of gain image)” hereinafter) obtained from the plurality of gain images.

The image processing unit 109 processes the radiation image obtained from the FPD 102. The image processing unit 109 includes, as functional components, a gain correction unit 110, a gain image generation unit 111, and an updating unit 112. These functional components may be implemented by the processor of the control unit 105 executing a predetermined program or, may be implemented using programs loaded from the storage unit 108 by one or a plurality of processors provided in the image processing unit 109. The processor in each of the control unit 105 and the image processing unit 109 is formed by, for example, a CPU (Central Processing Unit). The units of the image processing unit 109 may be formed by an integrated circuit or the like if the same functions can be obtained. In addition, the image processing apparatus 120 can be configured to include, as its internal components, a graphic control unit such as a GPU (Graphics Processing Unit), a communication unit such as a network card, and an input/output control unit such as a keyboard, a display, or a touch panel.

The monitor 106 (display unit) displays a radiation image (digital image) that the control unit 105 receives from the FPD 102, or an image processed by the image processing unit 109. The control unit 105 controls display of the monitor 106 (display unit). The operation unit 107 can input an instruction to the image processing unit 109 or the FPD 102, and accepts input of the instruction to the image processing unit 109 or the FPD 102 via a user interface. With this configuration, the radiation imaging system 100 can implement radiation imaging.

### Gain Correction Processing

### Gain Correction Assuming Linearity of Input/Output Characteristic of Pixel

Before an explanation of gain correction using a nonlinear function used in the first embodiment, gain correction (to be referred to as single-point gain correction hereinafter) assuming linearity of the input/output characteristic of a pixel (the input/output characteristic of an imaging element) will be described. Single-point gain correction is represented by

Gaincor_{(x,y)}=pic_{(x,y)}*α/Gain_{(x,y)} (1)

where Gaincor is an image (corrected image) after gain correction, and pie is a captured image. Gain is a gain image, and the gain image is an image captured without arranging the object 103. In each image, (x, y) represents a pixel. Gaincor_{(x,y)} is the pixel value in the image after gain correction, and pic_{(x,y)} is the pixel value in the captured image. Gain_{(x,y)} is the pixel value in the gain image representing the input/output characteristic. In Gaincor_{(x,y)} and pic_{(x,y)}, x indicates the column number of the pixel arranged in the two-dimensional area of the radiation detecting unit of the FPD 102, and y indicates the row number of the pixel.

α is the updating coefficient that adjusts the output value after pixel gain correction of the image after gain correction. As the updating coefficient α, an arbitrary value can be set. For example, the updating coefficient α can be calculated using the average value of the pixel values of the gain image or the dose at the time of obtaining the gain image. In the single-point gain correction, the input/output characteristic of the pixel is assumed to be linear. Hence, if the input/output characteristic of the pixel is nonlinear, as the deviation from the incident dose (imaging dose) at the time of capturing of the gain image becomes large, an error in gain correction (gain correction error) that correction cannot sufficiently be done by the gain correction may occur.

### Gain Correction Considering Nonlinearity of Input/Output Characteristic of Pixel

To reduce the gain correction error that can occur in single-point gain correction, gain correction for correcting a radiation image using a correction function (nonlinear function) obtained based on a plurality of gain images under different imaging doses is used. This gain correction will be referred to as multi-point gain correction hereinafter. In multi-point gain correction, as an interpolation method for interpolating the input/output characteristic of pixel, for example, an interpolation method such as linear interpolation, polynomial interpolation, or spline interpolation is used. In this embodiment, as polynomial interpolation, a method of correcting a radiation image by a cubic polynomial function like equation (2) using the input/output characteristic of a pixel in a gain image will be described. Note that the polynomial interpolation is not limited to the example to be described below, and the order, the coefficient, and the like of the polynomial function can be changed variously.

Gaincor_{(x,y)}=a_{(x,y)}×pic_{(x,y)}³+b_{(x,y)}×pic_{(x,y)}²+c_{(x,y)}×pic_{(x,y)} (2)

wherein Gaincor_{(x,y)} is the pixel value in the image (corrected image; Gaincor) after gain correction, in which the nonlinearity of the input/output characteristic is corrected for each pixel in the captured image. pic_{(x,y)} is the pixel value in the captured image. In Gaincor_{(x,y)} and pic_{(x,y)}, x indicates the column number of the pixel arranged in the two-dimensional area of the radiation detecting unit of the FPD 102, and y indicates the row number of the pixel. a_{(xy)}, b_{(x,y)}, and c_{(x,y)} are the gain data (gain coefficients) of orders in the cubic polynomial function. The gain coefficients a_{(x,y)}, b_{(x,y)}, and c_{(x,y)} of the orders can be obtained using the least square method or Gaussian elimination for the plurality of gain images under different imaging doses and output values after gain correction of the gain images.

The gain correction unit 110 corrects (gain correction) a radiation image using the input/output characteristic of a pixel, which represents the relationship between the dose of radiation on the pixel and the signal output from the pixel and is obtained using gain data (gain coefficients) based on a plurality of gain images obtained in advance under different doses. The gain correction unit 110 may perform correction by obtaining the gain coefficients a_{(x,y)}, b_{(x,y)}, and c_{(x,y)} of the orders for each pixel each time when performing gain correction, or may obtain the gain coefficients a_{(x,y)}, b_{(x,y)}, and c_{(x,y)} of the orders in advance, store these in the storage unit 108, and read out the gain coefficients a_{(x,y)}, b_{(x,y)}, and c_{(x,y)} from the storage unit 108 each time when performing gain correction.

In this way, the gain correction unit 110 can perform gain correction of the input/output characteristic of a pixel using the correction function (nonlinear function) of gain data (gain coefficients) obtained based on a plurality of gain images obtained in advance under different imaging doses.

In the radiation imaging system 100, however, since the arrangement relationship between the radiation tube 101 and the FPD 102, the irradiation distribution of the radiation tube 101, the light emission amount of phosphors in the radiation detecting unit of the FPD 102, and the like may change over time, the amounts of aging from the timing of gain image capturing may cause an error (gain correction error) in gain correction.

To reduce the gain correction error caused by the aging, for example, maintenance of the radiation imaging system 100 is conducted normally about once a year. This maintenance includes obtaining of a plurality of gain images to be used in multi-point gain correction. According to the multi-point gain correction, the gain correction error can be reduced, but the man-hours (time) required to obtain the plurality of gain images under different imaging doses can be enormous as compared to single-point gain correction.

### Estimation of Correction Function (Nonlinear Function) to be Used in Multi-Point Gain Correction

In the first embodiment of the present invention, the correction function (nonlinear function) to be used for gain images under different doses or in multi-point gain correction is estimated from a single (one) newly obtained gain image. This makes it possible to reduce the maintenance man-hours in multi-point gain correction required to obtain a plurality of gain images to about the same amount as the maintenance man-hours in single-point gain correction.

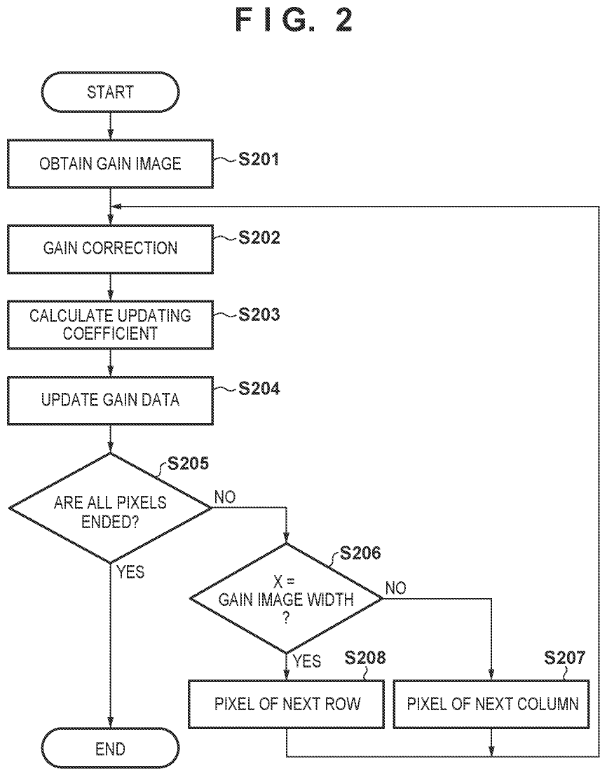

FIG. 2 is a flowchart for explaining the procedure of processing of estimating the gain data (gain coefficients) of the correction function (nonlinear function) to be used in multi-point gain correction according to the first embodiment from a newly obtained gain image. Processing of estimating (obtaining) the gain data (gain coefficients) of the correction function (nonlinear function) to be used in multi-point gain correction will be described below in detail with reference to FIG. 2.

In step S201, the FPD 102 (image obtaining unit) of the radiation imaging system 100 newly captures (obtains) a gain image without arranging the object 103. If radiation is emitted from the radiation tube 101 without arranging the object, the FPD 102 accumulates charges based on an image signal and obtains a gain image. To reduce noise, the gain image is captured a plurality of times under the same imaging dose. The gain image generation unit 111 executes averaging processing for the gain images captured a plurality of times and obtains an averaged gain image.

In step S202, the gain correction unit 110 executes gain correction (multi-point gain correction) for the gain image obtained in step S201. At the time of gain correction, the gain correction unit 110 obtains, from the storage unit 108, gain data (gain coefficients) obtained based on a plurality of gain images obtained in advance under different imaging doses. The gain correction unit 110 executes multi-point gain correction based on the obtained gain data (gain coefficients) and the gain image captured in step S201. Here, as the correction function (nonlinear function) used in multi-point gain correction, for example, the cubic polynomial function described concerning equation (2) can be used. The gain correction unit 110 executes multi-point gain correction for the gain image, thereby obtaining an image (corrected image: Gaincor) after gain correction.

Based on the corrected image obtained by the gain correction unit 110 correcting the gain image newly obtained by the FPD 102 (image obtaining unit) and a target pixel value γ of the corrected image, the updating unit 112 calculates an updating coefficient β_{(x,y)} and updates the gain data by the updating coefficient β_{(x,y)}. That is, the updating unit 112 updates the gain data by the updating coefficient obtained based on the corrected image obtained by the gain correction unit 110 correcting the gain image newly obtained by the FPD 102 (image obtaining unit) and the target pixel value of the corrected image.

In step S203, the updating unit 112 applies the result of multi-point gain correction (step S202) to equation (3), thereby calculating, for each pixel, the updating coefficient β_{(x,y)} for updating the gain data (gain coefficients) of the orders of the polynomial function for multi-point gain correction.

β_{(x,y)}=γ/Gaincor_{(x,y)} (3)

where γ is the target pixel value in the corrected image (Gaincor) after multi-point gain correction is performed based on the newly captured gain image. The target pixel value γ can be set using the average value of the pixel values in the gain image, the imaging dose used when obtaining the gain image, an output value estimated from the imaging dose, or the like. Also, in equation (3), Gaincor_{(x,y)} is a pixel value in the image (corrected image) after gain correction, which is obtained by the processing in step S202.

In step S204, using the updating coefficient β_{(x,y)} obtained in step S203, the updating unit 112 updates the gain data (gain coefficients) of the orders of the polynomial function to be used in multi-point gain correction based on

a_{(x,y)}=a_{(x,y)}×β_{(x,y)}

b_{(x,y)}=b_{(x,y)}×β_{(x,y)}

c_{(x,y)}=c_{(x,y)}×β_{(x,y)} (4)

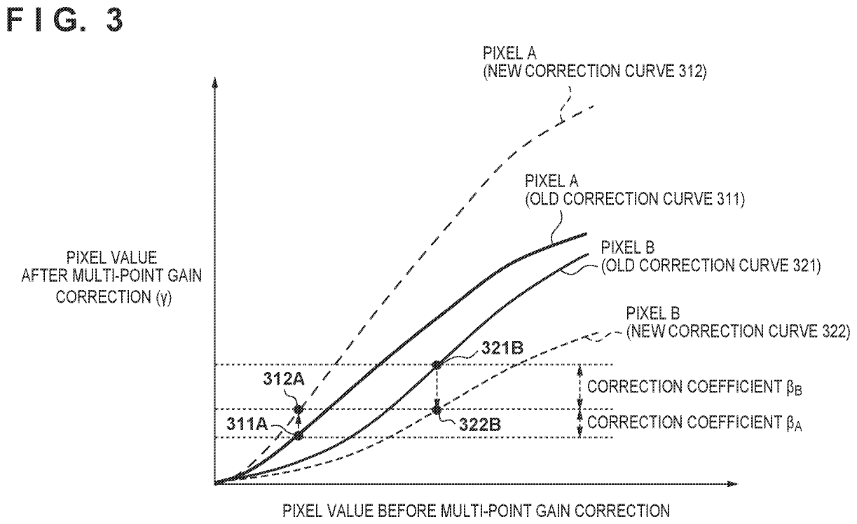

FIG. 3 is a graph exemplarily showing changes of pixel values caused by multi-point gain correction using the polynomial function whose gain data (gain coefficients) are updated based on the updating coefficient β_{(x,y)}. The abscissa represents a pixel value before multi-point gain correction, and the ordinate represents a pixel value after multi-point gain correction.

As an example of the pixels in the newly obtained gain image, FIG. 3 shows changes of a pixel A and a pixel B. In the pixel A and the pixel B, assuming that the tendency of the input/output characteristic of each pixel does not change, the input/output characteristic (an old correction curve indicated by a solid line) is maintained, and curve fitting (interpolation processing) is performed by multiplying the gain data (gain coefficients) of the orders by the updating coefficient β_{(x,y)}, thereby generating a new correction curve indicated by a broken line.

In FIG. 3, an old correction curve 311 of the pixel A represents the input/output characteristic of the pixel A by multi-point gain correction using a polynomial function whose gain coefficients of orders are not updated. In addition, a new correction curve 312 of the pixel A represents the input/output characteristic of the pixel A by multi-point gain correction using a polynomial function whose gain coefficients of orders are updated based on the updating coefficient β_{(x,y)}. Letting β_{A} be the updating coefficient of the pixel A, a pixel value 311A of the pixel A on the old correction curve 311 changes to a pixel value 312A of the pixel A on the new correction curve 312.

Also, in FIG. 3, an old correction curve 321 of the pixel B represents the input/output characteristic of the pixel B by multi-point gain correction using a polynomial function whose gain coefficients of orders are not updated. A new correction curve 322 of the pixel B represents the input/output characteristic of the pixel B by multi-point gain correction using a polynomial function whose gain coefficients of orders are updated based on the updating coefficient β_{(x,y)}. Letting β_{B} be the updating coefficient of the pixel B, a pixel value 321B of the pixel B on the old correction curve 321 changes to a pixel value 322B of the pixel B on the new correction curve 322.

The pixel value represented by the old correction curve 311 or 321 is multiplied by the ratio (updating coefficient β) of the target pixel value γ of the corrected image to the pixel value Gaincor_{(x,y)} of the corrected image Gaincor that has undergone multi-point gain correction using the polynomial function whose gain data (gain coefficients) are not updated, thereby updating the gain data (gain coefficients). The input/output characteristic of the pixel represented by the old correction curve 311 or 321 (solid line) is updated to the input/output characteristic of the pixel represented by the new correction curve 312 or 322 (dotted line). The calculation of the updating coefficient β_{(x,y)} explained concerning equation (4) is executed for each pixel of the newly captured gain image, and the gain data (gain coefficients) of the orders are updated based on the updating coefficient β_{(x,y)}, thereby reducing the gain correction error caused by aging such as the light emission distribution of phosphor or the radiation irradiation distribution.

In step S205, the updating unit 112 judges, for each pixel of the newly captured gain image, whether the gain coefficients of the polynomial function of gain correction are updated. If the gain coefficients are not updated for all pixels (NO in step S205), the updating unit 112 advances the process to step S206.

In step S206, the updating unit 112 refers to the column number (x) of the pixel that has undergone gain coefficient updating processing, and determines whether the gain coefficient updating processing is ended up to the array (x=N) corresponding to the gain image width (N pixels). If the updating processing is not ended (NO in step S206), the updating unit 112 advances the process to step S207.

In step S207, the updating unit 112 sets a pixel of the next column (x=x+1: the initial value is x=0) to the gain coefficient updating target and returns the process to step S202.

On the other hand, if it is determined in step S206 that the gain coefficient updating processing is ended up to the array (N) corresponding to the gain image width (N pixels) (YES in step S206), the updating unit 112 advances the process to step S208. The updating unit 112 sets a pixel of the next row (y=y+1: the initial value is y=0) to the gain coefficient updating target and returns the process to step S202.

In step S202, the gain correction unit 110 executes the same multi-point gain correction as the processing contents of step S202 described above for a pixel of the gain image in the column set in step S207 or the row set in step S208. In step S203, the updating unit 112 calculates the updating coefficient β_{(x,y)} based on the result of multi-point gain correction (step S202). In step S204, the updating unit 112 updates the gain data (gain coefficients) of orders in the polynomial function to be used in multi-point gain correction using the updating coefficient β_{(x,y)} obtained in step S203.

In step S205, the updating unit 112 determines, for all pixels of the newly captured gain image, whether the gain coefficients are updated. If the gain coefficients are not updated (NO in step S205), the updating unit 112 advances the process to step S206 to repeat the same processing as described above. On the other hand, if it is determined in step S205 that the gain coefficients are updated for all pixels (YES in step S205), the processing is ended. As described with reference to FIG. 2, the gain data (gain coefficients) of the orders in the polynomial function are updated for each pixel based on the newly obtained gain image, thereby estimating (obtaining) the correction function (nonlinear function) for multi-point gain correction, in which nonlinearity in the input/output characteristic of each pixel is corrected.

Note that in equation (4) described above, as the processing of estimating the correction function (nonlinear function) to be used in multi-point gain correction, an example in which the gain data (gain coefficients) of the correction function (nonlinear function) are updated using an updating coefficient has been described. In this embodiment, in addition to this example, for example, a plurality of gain images captured (obtained) in advance by the FPD 102 (image obtaining unit) under different doses can be held as gain data in the storage unit 108, and the updating unit 112 can update, based on the updating coefficient β_{(x,y)}, pixel values obtained based on the plurality of gain images captured (obtained) in advance and estimate a new gain image including the updated pixel values.

In this example, in step S204 of FIG. 2, using the updating coefficient obtained in step S203, the updating unit 112 updates a pixel value in the gain image based on

Gain1_{(x,y)}=Gain1_{(x,y)}×β_{(x,y)}

Gain2_{(x,y)}=Gain2_{(x,y)}×β_{(x,y)}

Gain3_{(x,y)}=Gain3_{(x,y)}×β_{(x,y)} (5)

where Gain1, Gain2, and Gain3 are gain images captured under different doses, and Gain1_{(x,y)}, Gain2_{(x,y)}, and Gain3_{(x,y)} represent pixel values in the gain images captured under the different doses. β_{(x,y)} is the updating coefficient obtained by equation (3). x indicates the column number of the pixel arranged in the two-dimensional area of the radiation detecting unit, and y indicates the row number of the pixel.

If pixel values obtained from three gain images as indicated by equation (5) are held in the storage unit 108 as gain data (the pixel values of the gain images), the updating unit 112 can estimate (obtain) a new gain image by updating the pixel values of the gain images using the updating coefficient β_{(x,y)}. Here, the gain image newly obtained to calculate the updating coefficient is a single (one) gain image, and the number is smaller than the number of the plurality of gain images (Gain1, Gain2, and Gain3) obtained in advance.

According to the first embodiment, the man-hours required to obtain a plurality of gain images can be reduced by updating gain data to be used in gain correction by an updating coefficient calculated using a newly obtained gain image. This makes it possible to reduce the maintenance man-hours in multi-point gain correction required to obtain a plurality of gain images to about the same amount as the maintenance man-hours in single-point gain correction, and prevent an increase in the maintenance man-hours, which is the problem of multi-point gain correction.

### Second Embodiment

In the first embodiment, processing of calculating the updating coefficient β_{(x,y)} using the result of multi-point gain correction of a newly obtained gain image and correcting the input/output characteristic of a pixel before correction (the old correction curve indicated by the solid line in FIG. 3) to the input/output characteristic after correction using the updating coefficient β_{(x,y)} (the new correction curve indicated by the broken line in FIG. 3) has been described. However, since not a few errors may occur in the multi-point gain correction, the errors may affect the updating coefficient β as well. In the second embodiment, a configuration for reducing the error of the updating coefficient β described in the first embodiment will be described.

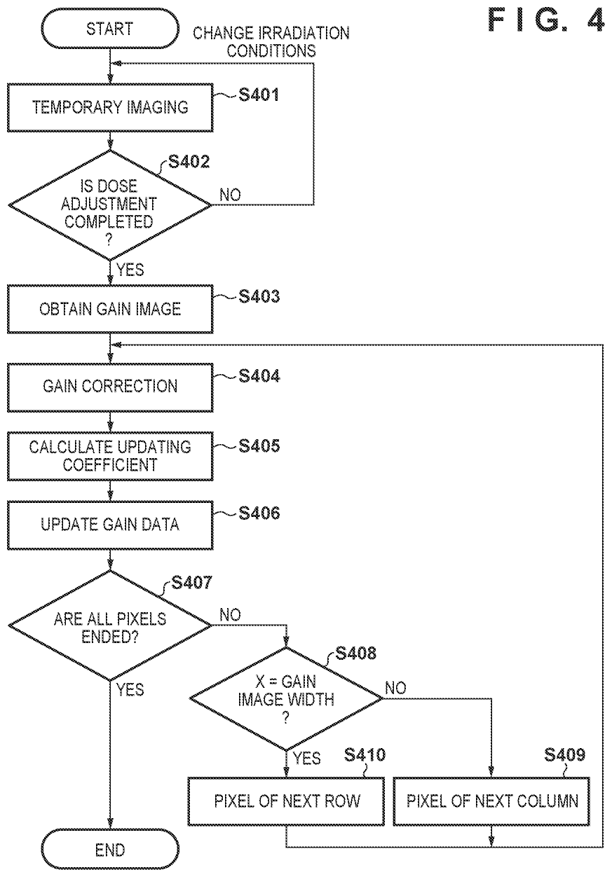

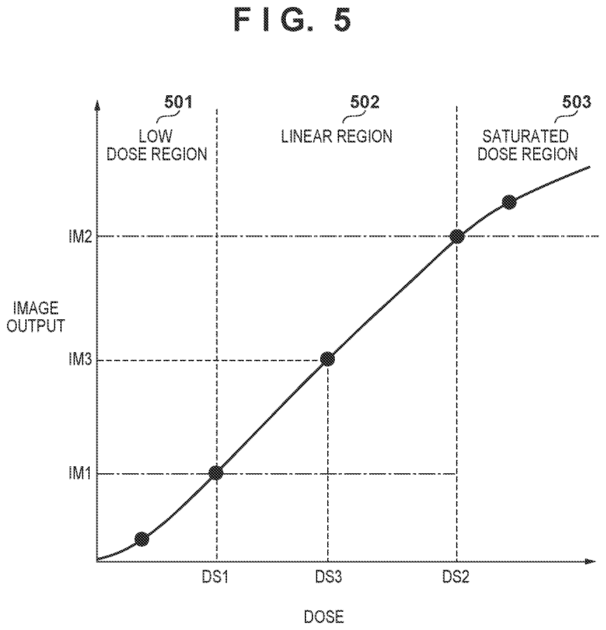

FIG. 4 is a flowchart for explaining the procedure of processing of estimating the gain data. (gain coefficients) of a correction function (nonlinear function) to be used in multi-point gain correction according to the second embodiment from a newly obtained gain image. In FIG. 4, the processes of steps S403 to S410 are the same as those of steps S201 to S208 in FIG. 2 of the first embodiment, and a detailed description thereof will be omitted. This embodiment is different from the first embodiment in that before a gain image is newly captured (obtained: step S403), temporary capturing of a gain image (step S401) and dose adjustment (step S402) are performed. FIG. 5 is a graph schematically showing the input/output characteristic of a pixel in each dose region according to the second embodiment. In FIG. 5, the abscissa represents a dose, and the ordinate represents an image output (pixel value). Processing according to the second embodiment will be described below with reference to FIGS. 4 and 5.

In step S401, a radiation imaging system 100 executes radiation imaging (temporary imaging) for deciding gain image capturing conditions without arranging an object 103 on an FPD 102.

In step S402, a control unit 105 calculates the average value of pixel values in a captured radiation image, and calculates an error to the target average value. Here, the control unit 105 calculates the average value of pixel values in the entire radiation image or in a ROI (Region Of Interest) as a part of the radiation image. The ROI as a part of the radiation image includes, for example, the center part (center ROI) of the radiation image.

If a plurality of gain images obtained in advance under different imaging doses are held in a storage unit 108, the target average value is set based on the average value of the pixel values of the plurality of gain images (for example, the pixel values in the center ROI).

As shown in FIG. 5, in a linear region 502, the change of the image output (the input/output characteristic of a pixel) to the dose exhibits linearity, and between a dose DS1 and a dose DS2, the image output changes from an image output IM1 to an image output IM2. In a low dose region 501 where the dose is lower than the dose DS1 or in a saturated dose region 503 where the dose is higher than the dose DS2, the change of the image output to the dose is large as compared to the linear region 502, and the error of correction by an updating coefficient β_{(x,y)} tends to be large.

For this reason, of the plurality of gain images under different doses, the gain image in the linear region is preferably used as the gain image used to calculate the target average value. In the example shown in FIG. 5, gain images captured in the range from the dose DS1 (first dose) at the lower limit of the linear region where the change of the image output to the dose exhibits linearity to the dose DS2 (second dose) at the upper limit of the linear region can be gain images in the linear region. In this case, a gain image captured under a dose DS3 (third dose) between the dose DS1 (first dose) and the dose DS2 (second dose) is also included in the gain images in the linear region. The control unit 105 sets the target average value based on the average value of the pixel values of the plurality of gain images obtained in advance under the different doses.

Here, let Gain1, Gain2, and Gain3 be the plurality of vain images captured under the doses DS1, DS2, and DS3. In this case, the control unit 105 calculates the target average value based on the plurality of gain images (Gain1, Gain2, and Gain3) captured in the range from the dose DS1 (first dose) at the lower limit of the linear region to the dose DS2 (second dose) at the upper limit of the linear region. Let IM1, IM2, and IM3 be the average values of the pixel values (the pixel values in the center ROI) of the plurality of gain images (Gain1, Gain2, and Gain3). In this case, the control unit 105 sets the target average value based on the average value in at least one of the plurality of gain images. For example, if all the plurality of gain images (Gain1, Gain2, and Gain3) are targets, the control unit 105 sets the average value of IM1, IM2 and IM3 as the target average value.

Note that if no gain image is held as gain data in the storage unit 108, and only gain coefficients a_{(x,y)}, b_{(x,y)}, and c_{(x,y)} of orders used in polynomial interpolation are held in the storage unit 108, the target average value or the target dose (dose range) is preferably separately held in the storage unit 108. The control unit 105 adjusts the dose such that the error between the average value of the pixel values in the temporarily captured radiation image and the target average value becomes a predetermined value or less.

If it is determined, by the determination processing of step S402, that the error (the absolute value of the difference) between the average value of the pixel values in the temporarily captured (obtained) radiation image (the average value in the center ROI) and the target average value is not the predetermined value or less (NO in step S402), the control unit 105 adjusts the dose by changing irradiation conditions such as the tube current and the radiation irradiation time such that the error becomes small, and returns the process to step S401.

In step S401, the radiation imaging system 100 executes radiation imaging (temporary imaging) again under the adjusted dose. By the determination processing of step S402, the control unit 105 determines whether the error is the predetermined value or less. Upon determining that the error between the average value of the pixel values in the radiation image captured under the adjusted dose condition (the average value in the center ROI) and the target average value is the predetermined value or less (YES in step S402), the control unit 105 advances the process to step S403.

Note that in the determination processing of step S402, as the index used to calculate the error, not only the average value of the pixel values in the radiation image captured in step S401 but also a measurement result obtained by a dose measuring unit (for example, an area dosimeter (DAP)) measuring the dose at the time of imaging may be used. The dose measuring unit measures the dose of irradiated radiation, and the control unit 105 adjusts the dose such that the dose falls within the range from the dose DS1 (first dose) corresponding to the lower limit of the region where the input/output characteristic is linear to the dose DS2 (second dose) corresponding to the upper limit of the region where the input/output characteristic is linear.

In this case, as the target dose (dose range), for example, the range from the dose DS1 (first dose) at the lower limit of the linear region to the dose DS2 (second dose) at the upper limit of the linear region, shown in FIG. 5, is held in the storage unit 108, and the control unit 105 compares the dose measurement result by the area dosimeter (DAP) with the target dose (dose range).

If the dose error (the absolute value of the difference) between the dose measurement result and the target dose is not a predetermined value or less (NO in step S402), the control unit 105 adjusts the dose by changing irradiation conditions such as the tube current and the radiation irradiation time such that the dose error becomes small, and returns the process to step S401. On the other hand, if it is determined, by the determination processing of step S402, that the dose error is the predetermined value or less (YES in step S402), the control unit 105 advances the process to step S403. In step S403, the FPD 102 (image obtaining unit) newly captures (obtains) a gain image without arranging the object 103. The FPD 102 (image obtaining unit) captures (obtains) the new gain image under a dose in the region (linear region 502) where the input/output characteristic is linear.

Processing (steps S403 to S410) from step S403 is the same as in the first embodiment, and a description thereof will be omitted.

According to this embodiment, when dose adjustment processing is performed before a gain image to be used in multi-point gain correction is newly captured (obtained) such that the change of the image output to the dose falls within the range of the linear region, the error generated by the calculation of the updating coefficient p can be reduced, and accurate multi-point gain correction can be performed by few maintenance man-hours.

According to the present invention, gain data to be used in gain correction is updated using an obtained updating coefficient, thereby reducing man-hours required to obtain a plurality of gain images.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention leas been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-100431, filed Jun. 16, 2021, which is hereby incorporated by reference herein in its entirety.

What is claimed is:

1. A radiation imaging system comprising:
an image obtaining unit including a radiation detecting unit in which pixels configured to output signals according to a dose of irradiated radiation are arranged in a two-dimensional area, and configured to obtain a radiation image based on the signals;
a correction unit configured to correct the radiation image using an input/output characteristic of a pixel, which represents a relationship between the dose of radiation on the pixel and the signal output from the pixel and is obtained using gain data based on a plurality of gain images obtained under different doses; and
an updating unit configured to update the gain data using an updating coefficient obtained based on the gain data and a gain image newly obtained by the image obtaining unit.

2. The system according to claim 1, wherein the updating unit updates the gain data using the updating coefficient obtained based on a corrected image obtained by the correction unit correcting the gain image newly obtained by the image obtaining unit and a target pixel value of the corrected image.

3. The system according to claim 2, wherein the updating unit updates the gain data by calculating, as the updating coefficient, a ratio of the target pixel value of the corrected image to a pixel value of the corrected image, and multiplying the gain data by the updating coefficient.

4. The system according to claim 1, wherein the correction unit corrects the newly obtained gain image by interpolating the input/output characteristic of the pixel by a nonlinear function.

5. The system according to claim 4, wherein interpolation using the nonlinear function includes one of polynomial interpolation and spline interpolation.

6. The system according to claim 4, wherein the gain data includes a coefficient of the nonlinear function.

7. The system according to claim 1, wherein the gain data includes pixel values obtained from a plurality of gain images obtained by the image obtaining unit under different doses.

8. The system according to claim 7, wherein the updating unit updates, based on the updating coefficient, the pixel values of the plurality of gain images obtained.

9. The system according to claim 7, wherein the number of newly obtained gain images is smaller than the number of the plurality of gain images obtained.

10. The system according to claim 1, wherein the image obtaining unit captures the new gain image under a dose in a region where the input/output characteristic is linear.

11. The system according to claim 1, further comprising a storage unit configured to hold the gain data obtained under a different dose,
wherein the correction unit obtains the gain data from the storage unit and performs the correction.

12. The system according to claim 1, further comprising a control unit configured to adjust the dose of the gain image to be newly obtained by the image obtaining unit,
wherein the updating unit updates the gain data using the updating coefficient obtained based on a corrected image obtained by the correction unit correcting the gain image newly obtained based on the adjusted dose and the target pixel value of the corrected image.

13. The system according to claim 12, wherein the control unit adjusts the dose such that an error between an average value of pixel values in a temporarily captured radiation image and a target average value is not more than a predetermined value.

14. The system according to claim 13, wherein the control unit sets the target average value based on an average value of pixel values of the plurality of gain images obtained under different doses.

15. The system according to claim 12, further comprising a dose measuring unit configured to measure the dose of the irradiated radiation,
wherein the control unit adjusts the dose such that the dose falls within a range from a first dose corresponding to a lower limit of a region where the input/output characteristic is linear to a second dose corresponding to an upper limit of the region.

16. An image processing apparatus for processing a radiation image obtained by an image obtaining unit including a radiation detecting unit in which pixels configured to output signals according to a dose of irradiated radiation are arranged in a two-dimensional area, and configured to obtain a radiation image based on the signals, comprising:
a correction unit configured to correct the radiation image using an input/output characteristic of a pixel, which represents a relationship between the dose of radiation on the pixel and the signal output from the pixel and is obtained using gain data based on a plurality of vain images obtained under different doses; and
an updating unit configured to update the gain data using an updating coefficient obtained based on the gain data and a gain image newly obtained by the image obtaining unit.

17. The apparatus according to claim 16, further comprising a control unit configured to adjust the dose of the gain image to be newly obtained by the image obtaining unit,
wherein the updating unit updates the gain data using the updating coefficient obtained based on a corrected image obtained by the correction unit correcting the gain image newly obtained based on the adjusted dose and the target pixel value of the corrected image.

18. An image processing method of processing a radiation image obtained by an image obtaining unit including a radiation detecting unit in which pixels configured to output signals according to a dose of irradiated radiation are arranged in a two-dimensional area, and configured to obtain a radiation image based on the signals, comprising:
correcting the radiation image using an input/output characteristic of a pixel, which represents a relationship between the dose of radiation on the pixel and the signal output from the pixel and is obtained using gain data based on a plurality of gain images obtained under different doses; and
updating the gain data using an updating coefficient obtained based on the gain data and a gain image newly obtained by the image obtaining unit.

19. The method according to claim 18, further comprising adjusting the dose of the gain image to be newly obtained by the image obtaining unit,
wherein in the updating, the gain data is updated using the updating coefficient obtained based on a corrected image obtained, in the correcting, by correcting the gain image newly obtained based on the adjusted dose and the target pixel value of the corrected image.

20. A non-transitory computer readable storage medium storing a program configured to cause a computer to execute an image processing method defined in claim 18.
